(19) United States
(12) Patent Application Publication
(10) Pub. No.: US 2002/0173710 A1
(43) Pub. Date: Nov. 21, 2002

(54) Sensor for biopotential measurements

(76) Inventor: Mark J. Licata, Doswell, VA (US)

Correspondence Address: Mark J. Licata, 11504 Riveredge Rd., Doswell, VA, 23047, US

(21) Appl. No.: 09/859,307

(22) Filed: May 18, 2001

(51) Int. Cl. A61B 5/04

(52) U.S. Cl. 600/397

(57) ABSTRACT

A sensor for biopotential measurements is designed to detect low voltage electrical signals on a subject's skin surface. A plurality of soft elastomeric bristles are arranged about the surface of the skin. Various bristles contain a wick, made of polyolefin, polyester or nylon, extending along its center axis with one end protruding from the bristle and another end in contact with a fluid reservoir. The wick is saturated with an electrically conductive liquid, such as a salt solution. The solution may contain a surfactant. The surface properties of the electrically conductive liquid and wick are optimized for predictable flow through the wick onto the skin surface. An electrode is positioned in the vicinity of the wick and the reservoir.

### TECHNICAL FIELD

This invention relates generally to the field of sensors for measuring electrical potentials obtained from the surface of the skin, for example, electroencephalogram (EEG), electrocardiogram (ECG), or electromyogram (EMG).

### BACKGROUND OF THE INVENTION

In the past, electroencephalogram (EEG), electrocardiogram (ECG), and electromyogram (EMG) electrodes are constructed as either a reusable or disposable device. The reusable electrodes are typically made of a Ag/AgCl disc encased in a plastic cover. These discs are applied to the skin of a subject after the skin is prepared with an abrasive and a conductive gel is applied to increase conductance. This type of electrode requires the assistance of technicians for proper use, and has been relegated for use in clinical environments. Disposable gel electrodes are also available, but are designed in such a way as to stick to the subject's hair. These disposable electrodes are therefore generally used on areas of the body without hair, or require the subject shave the area that the electrode will be applied to. With the advent of new modern electronic devices, there has developed a need for an electrode sensor that patients may use at home. These new devices allow patients to use new portable medical devices that require electrodes. The electrode needs to be non interfering with the patients hair and needs to be designed so that its use does not require chemicals or gels that can leave a mess. The prior art does not satisfy these requirements.

Conventional sensor configurations fail to disclose at least a single significant attribute of the present invention. What is needed is an electrode which may be used on skin with or without hair, does not require the use of external gels or creams to obtain adequate electrical conduction to the skin surface, may be comfortably worn for long periods of time, and may be properly applied by an individual's scalp without the assistance of a technician.

### BRIEF SUMMARY OF THE INVENTION

It is an object of this invention to describe a sensor for biopotential measurements comprising at least one elastomeric bristle having a base and a tip with a channel running there between and a porous wick extending through the channel, the tip contacting a skin surface; a reservoir containing an electrically conductive material is formed at the base of said elastomeric bristle; and an electrode for detecting electrical potential. The porous wick transports the electrically conductive material from the reservoir to the elastomeric bristle tip in order to conduct an electrical signal obtained from the skin surface, moisten the skin surface, and reduce the electrical resistance of the skin surface.

In yet a further aspect of the invention, a sensor for biopotential measurements wherein the reservoir is formed of at least one of: a porous material; and a hollow vessel capable of holding an electrically conductive liquid. The chemical properties of the electrically conductive liquid may be optimized for predictable flow through the porous wick onto the skin surface.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and form a part of the specification, illustrate an embodiment of the present invention and, together with the description, serve to explain the principles of the invention.

FIG. 1 is a cross-sectional view of an individual elastomeric bristle according to an embodiment of the present invention.

FIGS. 2A and 2B are exterior and interior views, respectively, of a surface comprising a plurality of elastomeric bristles with wicks in accordance with an embodiment of the present invention.

FIG. 3 is a cross-sectional view of an individual elastomeric bristle showing an electrode embedded in the elastomeric bristle according to an embodiment of the present invention.

FIG. 4 is a cross-sectional view of an aspect of an embodiment of the present invention showing an electrode and electrode cap fastened to a sensor top.

FIG. 5 is an external view of a sensor according to an embodiment of the present invention.

### DETAILED DESCRIPTION OF THE INVENTION

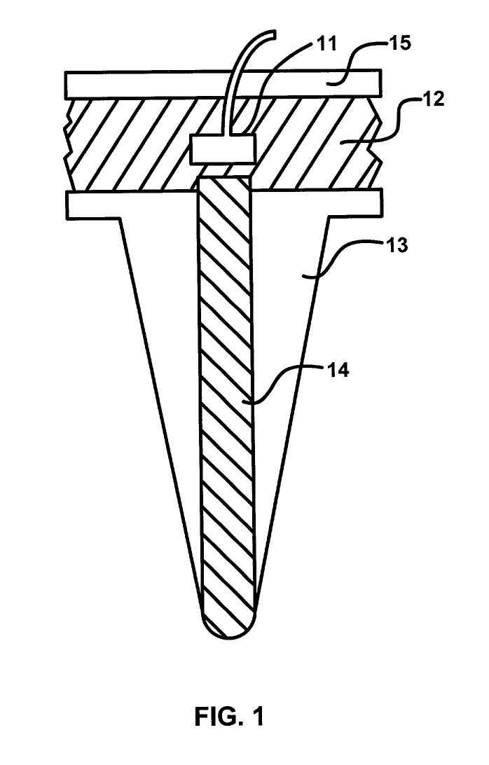

FIG. 1 is a cross-sectional view of an individual elastomeric bristle according to an embodiment of the present invention. As shown, a soft elastomeric bristle 13 contains a wick 14 of suitable material that extends through a channel in the center of the bristle 13. One end of the wick 14 protrudes from the end of the elastomeric bristle 13 to contact a skin surface. The other end of the wick 14 extends past the elastomeric bristle 13 into a fluid reservoir area 12. The fluid reservoir preferably has a sensor top 15 capping it. In the preferred embodiment, the wick material is polyolefin, but other materials are suitable including polyester or nylon.

The wick 14 may be saturated with an electrically conductive liquid, such as a solution of 0.2 to 1.0 molar sodium chloride, potassium chloride, sodium bicarbonate, or other salt solution. The solution serves to conduct the electrical signal obtained from the skin surface to an electrode 11 in the fluid reservoir area 12. The solution may also serve to moisten the skin surface and reduce the electrical resistance of the skin. The solution may also contain a surfactant to facilitate skin moistening, for example, 5 g/liter of sorbitan laurate.

The fluid reservoir 12 may be composed of a porous material capable of holding sufficient solution for the life of the sensor. Alternatively, the fluid reservoir 12 may be a hollow vessel to contain a volume of electrically conductive solution. The wick 14 conducts the solution to the skin surface as the fluid reservoir 12 is gradually depleted. When the fluid reservoir is fully depleted, it may be refilled by a variety of methods including reverse capillary action.

The chemical characteristics of the electrically conductive liquid may be manipulated by selecting specified components to form the electrically conductive liquid's composition. Particular materials may be mixed to create a solution of electrically conductive liquid with a specific surface energy. Additionally, various wick materials may exhibit different capillarity. In constructing the present invention, the composition of the electrically conductive liquid and the wick material may be predetermined for optimum control of the flow rate of the electrically conductive liquid through the wick 14. Flow control preferably determines the amount of skin surface wetting. Optimization of the rate of capillary action and surface energy may be performed to compensate for common chemical products applied to the hair and scalp, such as tonics, dyes, sprays and gels, which may react with the components of the sensor.

Alternatively, the fluid reservoir 12 may also be a volume of porous material loaded with a solution that is in fluidic contact with the wick 14. The material may be of such suitable material as cellulose or nylon.

At the bottom of the fluid reservoir 12, or at the junction of the wick 24 and porous reservoir material, an electrode 11 may be placed to detect the electrical potential conducted through the wick 14. The electrode 11 may be connected to instrumentation capable of amplifying and processing the electrical signal. The electrode 11 may be composed of any electrically conductive material, such as a combination of Ag and AgCl.

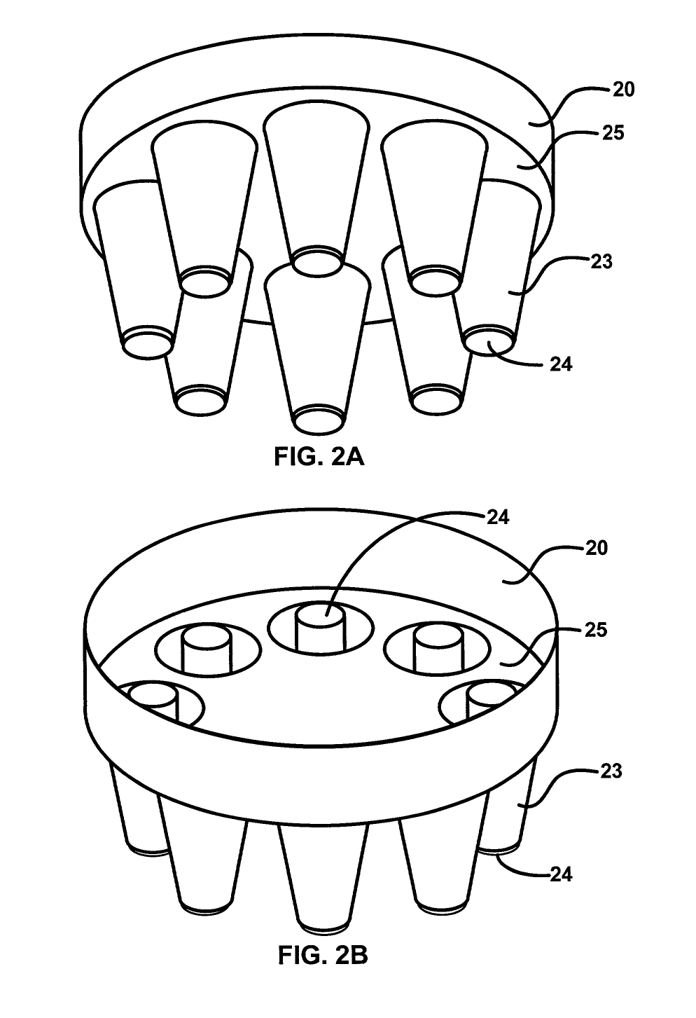

FIGS. 2A and 2B are exterior and interior views, respectively, of a surface comprising a plurality of elastomeric bristles with wicks in accordance with an embodiment of the present invention. As illustrated, a plurality of elastomeric bristles 23 may be physically linked to form a comb 25. The comb 25 is preferably made of a stiff but flexible material such as molded silicon rubber. Each of the elastomeric bristles 23 contains a wick 24 at its core. Each wick 24 may be coupled to a fluid reservoir 12 bound by an outer wall 20. The electrical signals obtained from the elastomeric bristles 23 may be summed in the fluid reservoir 12.

Experimentation has determined that it is not required that every elastomeric bristle 23 on the comb 25 be electrically conductive. In order to achieve a good measurement of biopotential and provide a sensor that is comfortable and securely applied to a skin surface, yet reduce complexity of the device and cost of manufacturing, the comb 25 may be formed with several of the elastomeric bristles 23 as “dummy” bristles that do not provide any electrical conductivity.

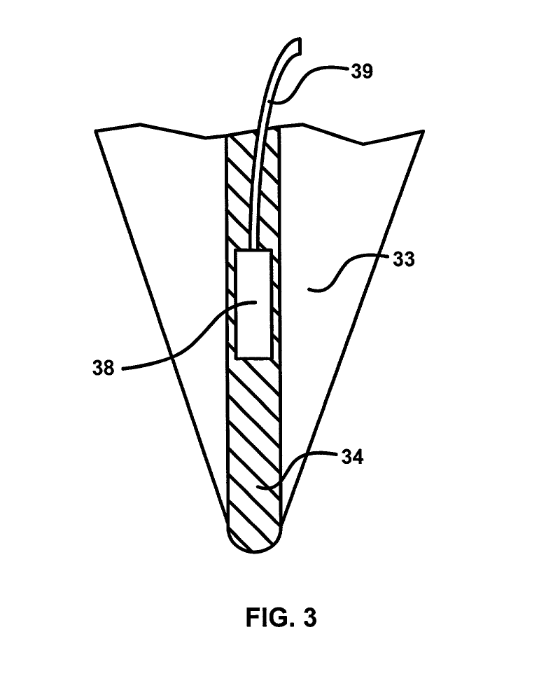

FIG. 3 is a cross-sectional view of an individual elastomeric bristle 33 showing an electrode 38 embedded in the elastomeric bristle 33 according to an embodiment of the present invention. In this embodiment, the conductive core 34 of the elastomeric bristle 33 is comprised of a porous wicking material. An electrode lead 39 may be used to conduct the signal out of the sensor assembly.

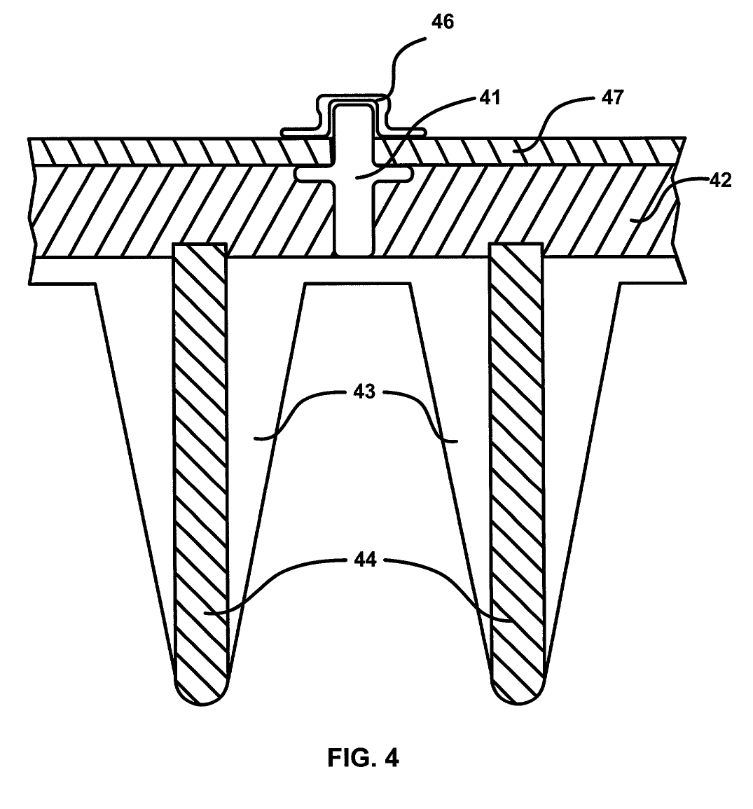

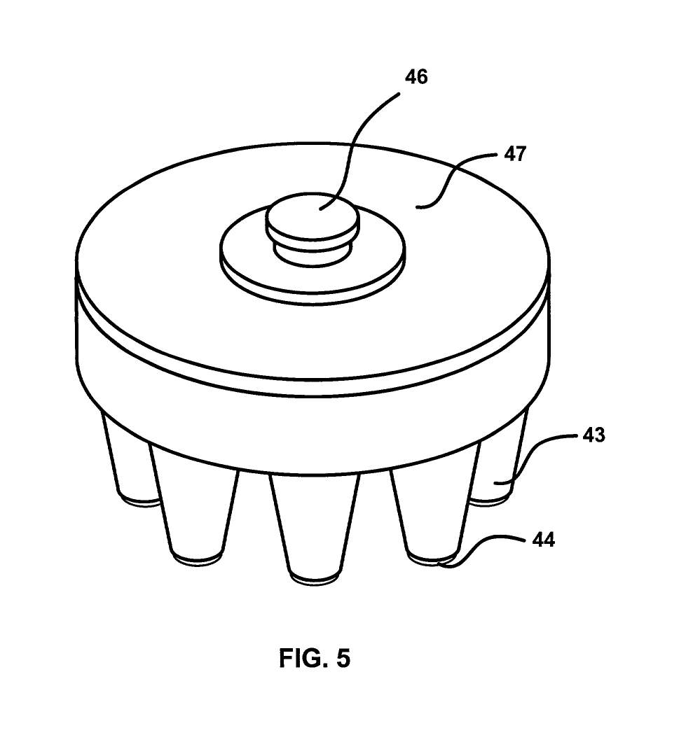

FIG. 4 is a cross-sectional view of an aspect of an embodiment of the present invention showing an electrode 41 and electrode cap 46 fastened to a sensor top 47. In this embodiment, the biopotential signals are conducted up the conductive cores 44 from each of the bristles 43 and are preferably summed in the reservoir 42. FIG. 5 is an external view of a sensor according to the embodiment illustrated in FIG. 4.

For any of the disclosed embodiments of the present invention, the sensor assembly may be disposable like a pen or an ink cartridge for a printer. This allows change over for different users or replacement.

The foregoing descriptions of the preferred embodiments of the present invention have been presented for purposes of illustration and description. They are not intended to be exhaustive or to limit the invention to the precise forms disclosed, and obviously many modifications and variations are possible in light of the above teaching. The illustrated embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A sensor for biopotential measurements comprising:
a. at least one elastomeric bristle having a base and a tip with a channel running therebetween and a porous wick extending through the channel, said tip contacting a skin surface;
b. a reservoir containing an electrically conductive material is formed at the base of said elastomeric bristle; and
c. an electrode for detecting electrical potential;
wherein, said porous wick transports the electrically conductive material from said reservoir to said elastomeric bristle tip in order to conduct an electrical signal obtained from the skin surface, moisten the skin surface, and reduce the electrical resistance of the skin surface.

2. A sensor for biopotential measurements according to claim 1 wherein said reservoir is formed of at least one of:
a. a porous material; and
b. a hollow vessel capable of holding an electrically conductive liquid.

3. A sensor for biopotential measurements according to claim 2 wherein the surface energy properties of the electrically conductive liquid are optimized for predictable flow through the porous wick onto the skin surface.

4. A sensor for biopotential measurements according to claim 2 wherein said porous material is selected from the group consisting of:
(a) cellulose; and
(b) nylon.

5. A sensor for biopotential measurements according to claim 2 wherein said electrically conductive liquid is a solution of 0.2 to 1.0 molar salt solution.

6. A sensor for biopotential measurements according to claim 5 wherein said salt solution is selected from the group consisting of:
a. sodium chloride;
b. potassium chloride; and
c. sodium bicarbonate.

7. A sensor for biopotential measurements according to claim 5 wherein said salt solution further contains 5 g/liter of sorbitan laurate as a surfactant.

8. A sensor for biopotential measurements according to claim 2 wherein said electrode is an electrically conductive coating on said porous material of the reservoir.

9. A sensor for biopotential measurements according to claim 8 wherein said electrically conductive coating is a composition comprising Ag and AgCl.

10. A sensor for biopotential measurements according to claim 1 wherein said electrode is placed at the junction of the porous wick and the reservoir.

11. A sensor for biopotential measurements according to claim 1 wherein said electrode is placed inside the elastomeric bristle channel.

12. A sensor for biopotential measurements according to claim 1 wherein said porous wick is made of a material selected from the group consisting of:
a. polyolefin;
b. polyester; and
c. nylon.

13. A sensor for biopotential measurements according to claim 1 wherein:
a. a plurality of elastomeric bristles are arranged about a surface and internally coupled to said reservoir; and
b. the biopotential signals obtained from each of said plurality of the bristles are summed in said reservoir.

14. A sensor for biopotential measurements according to claim 13 wherein at least three elastomeric bristles are arranged about said surface.

15. A sensor for biopotential measurements according to claim 1 wherein said electrode is made of a composition comprising Ag and AgCl.

16. A sensor for biopotential measurements comprising:
a. a plurality of physically linked elastomeric bristles; and
b. an electrode for detecting electrical potential;
wherein said plurality of elastomeric bristles comprise at least two different kinds of bristles.

17. A sensor for biopotential measurements according to claim 16 wherein a first kind of elastomeric bristle comprises a base and a tip with a channel running therebetween, said tip contacting a skin surface; and a second kind of elastomeric bristle which is non-conducting.

18. A sensor for biopotential measurements according to claim 17 further comprising:
a. a porous wick, the porous wick extending through the channel of said first kind of elastomeric bristles; and
b. a reservoir containing an electrically conductive liquid formed at the base of said elastomeric bristle;
wherein, said porous wick transports the electrically conductive liquid from said reservoir to said elastomeric bristle tip in order to: conduct an electrical signal obtained from the skin surface, moisten the skin surface, and reduce the electrical resistance of the skin surface.

19. A sensor for biopotential measurements according to claim 18 wherein said wicking material further contains a surfactant.

20. A sensor for biopotential measurements according to claim 16 wherein said electrode is formed at the base of said plurality of elastomeric bristles.

21. A sensor for biopotential measurements according to claim 20 wherein said electrode is made of a composition comprising Ag and AgCl.

22. A sensor for biopotential measurements according to claim 20 wherein said electrode comprises conductive spikes.

23. A sensor for biopotential measurements according to claim 20 wherein said electrode comprises a conductive disk.
